(19) United States
(12) Patent Application Publication
(10) Pub. No.: US 2025/0058020 A1
(43) Pub. Date: Feb. 20, 2025

(54) GELS AND RELATED METHODS FOR TREATING FISTULAS

(71) Applicant: Boston Scientific Scimed, Inc., Maple Grove, MN (US)

(72) Inventors: Robert COUGHLAN, Cork (IE); Mark LOONEY, Cork (IE); Patrick NUGENT, Cork (IE); Edward Joseph DEVLIN, Cork (IE); John MURPHY, Cork (IE); Megan CHROBAK, Westford, MA (US); Darragh O’DONOVAN, Edinburgh (GB); Conor O’SULLIVAN, Cork (IE)

(73) Assignee: Boston Scientific Scimed, Inc., Maple Grove, MN (US)

(21) Appl. No.: 18/807,099

(22) Filed: Aug. 16, 2024

Related U.S. Application Data
(60) Provisional application No. 63/520,407, filed on Aug. 18, 2023.

(51) Int. Cl. A61L 27/24; A61L 27/18; A61L 27/52; A61L 27/58

(52) U.S. Cl. CPC A61L 27/24; A61L 27/18; A61L 27/52; A61L 27/58; A61L 2430/34

(57) ABSTRACT

The present disclosure includes kits, compositions, and methods useful in medical procedures, such as treating a fistula or other target site of a subject. In some examples, the kit may comprise a dry particulate mixture, a first pharmaceutically acceptable buffer solution having a pH ranging from about 3 to about 5, and a second pharmaceutically acceptable buffer solution having a pH ranging from about 9 to about 11. The dry particulate mixture may comprise a plurality of polyethylene glycol particles and a plurality of collagen particles.

### CROSS-REFERENCE TO RELATED APPLICATIONS

This application claims the benefit of priority to U.S. Provisional Application No. 63/520,407, filed on Aug. 18, 2023, which is incorporated by reference herein in its entirety.

### TECHNICAL FIELD

This disclosure relates generally to gel materials useful for medical procedures, including endoscopic procedures.

### BACKGROUND

Gel materials are useful in a number of medical contexts to separate tissues and/or cover wounded tissue. A fistula is an abnormal connection or passageway that connects two organs or vessels that do not usually connect. Fistulas can develop between an intestine and the skin, between the vagina and the rectum, and within the gastrointestinal tract (e.g., stomach, intestine, colon, etc.), among other places. Adherence to tissue and ability to permanently close the fistula are challenging. Further, formulating a gel to promote tissue repair at the target site while maintaining bioresorbability of the gel can be difficult.

### SUMMARY

The present disclosure includes kits, compositions, and methods useful in medical procedures. For example, the present disclosure includes a kit comprising a dry particulate mixture, a first pharmaceutically acceptable buffer solution having a pH ranging from about 3 to about 5, and a second pharmaceutically acceptable buffer solution having a pH ranging from about 9 to about 11. The dry particulate mixture may comprise a plurality of polyethylene glycol particles and a plurality of collagen particles. The first pharmaceutically acceptable buffer solution may comprise at least one crosslinking agent, optionally wherein the crosslinking agent comprises trilysine acetate, N-(3-dimethylaminopropyl)-N′-ethylcarbodiimide hydrochloride, or N-hydroxysuccinimide. The weight ratio of the plurality of polyethylene glycol particles to the plurality of collagen particles in the dry particulate mixture may range from about 8:1 to about 6:1. The plurality of collagen particles may have an average particle size ranging from about 300 μm to about 500 μm. The plurality of polyethylene glycol particles may comprise chemically-modified polyethylene glycol, such as a polyethylene glycol polymer comprising one or more functional groups chosen from carboxylic acid groups, ester groups, amine groups, or a combination thereof. Optionally, the one or more functional groups may comprise succinimide groups. The first pharmaceutically acceptable buffer solution and/or second pharmaceutically acceptable buffer solution may comprise a radiopaque material and/or an antimicrobial agent. In some examples, the kit may further comprise instructions for combining the dry particulate mixture with the first and second pharmaceutically acceptable buffer solutions to form a gel for application to a fistula. Additionally or alternatively, the kit may further comprise a double-barreled syringe and instructions for combining the dry particulate mixture with the first pharmaceutically acceptable buffer solution to form a slurry, introducing the slurry into a first barrel of the syringe, and introducing the second pharmaceutically acceptable buffer solution into a second barrel of the syringe. Also disclosed herein are uses of the kit to treat a fistula of a subject, wherein the kit may be described above or elsewhere herein.

The present disclosure also includes methods of preparing the kit described above or elsewhere herein. The methods may comprise preparing a collagen scaffold, optionally wherein the collagen scaffold has an average pore size ranging from about 100 μm to about 200 μm, grinding the collagen scaffold to obtain the plurality of collagen particles, combining the plurality of collagen particles with the plurality of polyethylene glycol particles to obtain the dry particulate mixture, packaging the dry particulate mixture with the first pharmaceutically acceptable buffer solution and the second pharmaceutically acceptable buffer solution.

The present disclosure also includes methods of treating a subject. In some examples, the method comprises combining a dry particulate mixture with a first pharmaceutically acceptable buffer solution having a pH ranging from about 3 to about 5 to form a slurry and combining the slurry with a second pharmaceutically acceptable buffer solution having a having a pH ranging from about 9 to about 11 at a target site of the subject to form a gel at the target site. The dry particulate mixture may comprise a plurality of polyethylene glycol particles and a plurality of collagen particles. In some examples, the method comprises administering, to a target site of the subject, a slurry comprising a plurality of polyethylene glycol particles, a plurality of collagen particles, a first pharmaceutically acceptable buffer solution having a pH ranging from about 3 to about 5 and administering, to the target site, a second pharmaceutically acceptable buffer solution having a pH ranging from about 9 to about 11 to combine with the slurry. In some examples, the slurry and the second pharmaceutically acceptable buffer solution may form a gel at the target site, e.g., within 30 seconds of being combined at the target site. In some examples, the slurry and the second pharmaceutically acceptable buffer solution may be administered to the target site at the same time. The slurry and the second pharmaceutically acceptable buffer solution may be administered using a double-barreled syringe, e.g., wherein a first barrel of the syringe comprises the slurry and a second barrel of the syringe comprises the second pharmaceutically acceptable buffer solution.

### BRIEF DESCRIPTION OF THE FIGURES

The accompanying drawings, which are incorporated in and constitute a part of this application, illustrate various exemplary embodiments and together with the description, serve to explain the principles of the disclosed embodiments.

FIG. 1 shows a schematic depiction of a reaction mechanism for synthesizing an exemplary chemically-modified PEG, according to some aspects of the present disclosure; and

FIG. 2 shows a schematic depiction of a reaction mechanism for synthesizing another exemplary chemically-modified PEG, according to some aspects of the present disclosure.

FIGS. 3A and 3B show exemplary components of kits, according to some aspects of the present disclosure.

### DETAILED DESCRIPTION

Particular aspects of the present disclosure are described in greater detail below. The terms and definitions provided herein control, if in conflict with terms and/or definitions incorporated by reference.

As used herein, the terms “comprises,” “comprising,” or any other variation thereof are intended to cover a non-exclusive inclusion, such that a process, method, composition, article, or apparatus that comprises a list of elements does not include only those elements, but may include other elements not expressly listed or inherent to such process, method, composition, article, or apparatus. The term “exemplary” is used in the sense of “example” rather than “ideal.”

As used herein, the singular forms “a,” “an,” and “the” include plural reference unless the context dictates otherwise. The terms “approximately” and “about” refer to being nearly the same as a referenced number or value. As used herein, the terms “approximately” and “about” should be understood to encompass ±5% of a specified amount or value. All ranges are understood to include endpoints, e.g., a particle size ranging from 300 μm to 500 μm includes 300 μm, 500 μm, and all values between.

The present disclosure includes compositions, kits, and methods useful in medical procedures, such as treating a fistula (e.g., a fistula of a gastrointestinal tract) or other target site of a subject. The compositions, kits, and methods herein may be useful in endoscopic fistula closure procedures and endoscopic fistula ablation procedures.

In some examples, the compositions and kits herein may be combined and/or administered at a target site (e.g., fistula) of the subject to form a gel at the target site. The gel may adhere to tissue (e.g., mucosa and/or submucosa) of the target site, and may facilitate and/or promote regeneration of damaged tissue while also inhibiting or otherwise preventing the ingress of undesired material(s) to the target site.

### Compositions

The compositions herein may be useful in various medical procedures, such as endoscopic procedures, that include fistula closure and/or removal of fistula openings.

The compositions herein (e.g., gels such as hydrogels) may comprise a polyethylene glycol (PEG) polymer and collagen. For example, the gel may be prepared from a plurality of PEG particles, a plurality of collagen particles, and one or more pharmaceutically acceptable buffer solutions, e.g., a first pharmaceutically acceptable buffer solution and a second pharmaceutically acceptable buffer solution. The buffer solutions may have different pH values. For example, the PEG particles and the collagen particles may first be combined with a buffer having an acidic pH. Once combined with a buffer having a basic pH to act as an accelerant, the PEG particles form a gel that includes collagen, wherein the gel forms relatively quickly. When the gel is formed in situ at a target site, the gel may maintain the integrity of the area while the collagen promotes accelerated tissue regrowth and/or vascularization of tissue at the target site. Characteristics of the PEG polymer and collagen may be selected based on the target site(s) and type(s) of tissue to be treated.

For example, PEG polymers useful for the present disclosure may have an average molecular weight ranging from about 4,000 Daltons (Da) to about 16,500 Da, such as about 5,000 Da to about 12,000 Da, about 8,500 Da to about 16,500 Da, about 9,000 Da to about 16,000 Da, or about 10,000 Da to about 15,000 Da. PEG molecular weight may affect crosslinking density and the viscosity and/or strength of the gel.

The PEG polymer may comprise various functional groups, which may be capable of participating in crosslinking. For example, the PEG particles used to prepare the gel may comprise chemically-modified PEG. Exemplary functional groups include, but are not limited to, carboxylic acid groups, ester groups, and amine groups (including, e.g., succinimide groups). In some examples, the PEG polymer comprises one or more functional groups chosen from carboxylic acid groups, ester groups, amine groups, or a combination thereof. In at least one example, the PEG polymer comprises succinimide groups (e.g., N-hydroxysuccinimide) For example, the PEG polymer may comprise PEG-succinimidyl glutarate (PEG-SG). Optionally, one or more functional groups of the PEG particles may be radiopaque. For example, the PEG polymer may comprise a radiopaque functional group comprising iodine (e.g., 3,4,5-triiodobenzoic acid).

The PEG polymer may have a branched structure, such as a multi-arm structure. For example, the PEG polymer may have 3 to 10 arms, e.g., 5 to 8 arms, or 6 to 9 arms. Multi-armed PEG polymers include a central PEG chain and a plurality of arms bonded to the central chain. Each arm may have a functional group, which may be the same or different from the functional group of another arm. In at least one example, the PEG particles comprise a multi-armed PEG that comprises at least 3 arms, e.g., 3 to 10 arms, each arm comprising a succinimide group. Optionally, one or more arms may comprise a radiopaque moiety, e.g., a radiopaque functional group comprising iodine.

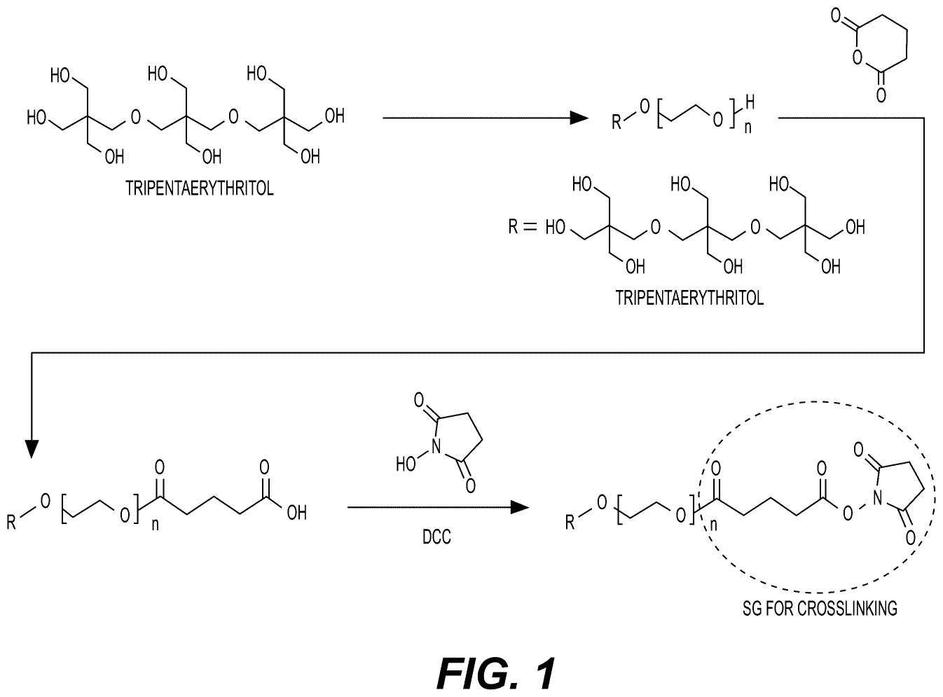

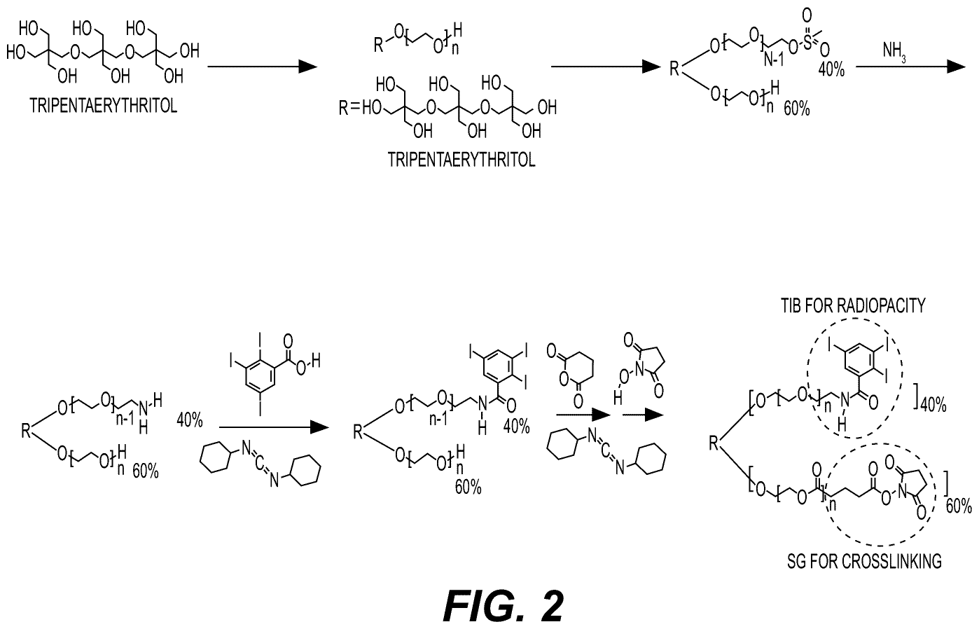

FIGS. 1 and 2 show reaction schemes for exemplary PEG polymers useful for the compositions, kits, and methods herein. For example, FIG. 1 shows synthesis of a PEG polymer that includes a succinimidyl glutarate group available for crosslinking to form a gel. FIG. 2 shows synthesis of a branched PEG polymer with a succinimidyl glutarate group available for crosslinking and a triiodobenzoate group for radiopacity.

Collagen particles useful for the present disclosure may comprise any suitable type of collagen including, e.g., Type I collagen, Type II collagen, Type III collagen, Type V collagen, and combinations thereof. In some examples, the collagen may be crosslinked. Crosslinking may provide advantages to the composition, such as improved bulk mechanical properties (e.g., hardness). Exemplary crosslinking agents include, but are not limited to, N-(3-dimethylaminopropyl)-N′-ethylcarbodiimide hydrochloride, N-hydroxysuccinimide, and combinations thereof.

As discussed below, the collagen particles may be prepared by grinding a scaffold into the desired particle size. For example, the collagen particles may have an average particle size ranging from about 200 μm to about 600 μm, such as about 250 μm to about 550 μm, or about 300 μm to about 500 μm. Particle size may be measured by a suitable instrument such as a Malvern Morphologi 4 instrument that uses static automated imaging. As used herein and for the purposes of this disclosure, the term “average particle size” refers to the circle equivalent (CE) diameter, which is defined as the diameter of a circle with the same area as the particle. The collagen particles may be porous, the porosity being retained in the gel composition. The size of the pores of the collagen may facilitate tissue repair. For example, the pore size may be on the order of the size of biological components involved in wound healing and/or tissue repair, such as fibroblasts and endothelial cells. Porosity of the collagen particles may be controlled during manufacture of the particles, e.g., via a porous collagen scaffold as discussed below.

The plurality of PEG particles and the plurality of collagen particles may be combined into a mixture, e.g., a dry particulate mixture or a mixture that includes a buffer solution in the form of a slurry. The weight ratio of the plurality of PEG particles to the plurality of collagen particles in the dry particulate mixture or slurry may range from about 10:1 to about 4:1, such as from about 8:1 to about 6:1, or from about 7:1 to about 5:1. In at least one example, the dry particulate mixture or slurry comprises from about 2.0 g to about 2.5 g of PEG polymer and from about 0.1 g to about 0.5 g collagen. In at least one example, the dry particulate mixture or slurry comprises from about 2.2 g to about 2.4 g PEG polymer and from about 0.3 g to about 0.4 g collagen.

Pharmaceutically acceptable buffer solutions useful for the present disclosure include, for example, phosphate buffers and borate buffers. The kits herein may comprise at least one, e.g., two pharmaceutically acceptable buffer solutions of different pH values. According to some aspects of the present disclosure, a first pharmaceutically acceptable buffer solution may have an acidic pH and a second buffer solution may have a basic pH. For example, a first pharmaceutically acceptable buffer solution may have a pH ranging from about 3 to about 5, such as a pH of about 4. The first pharmaceutically acceptable buffer solution may be pH adjusted with a suitable acid (e.g., hydrochloric acid).

A second pharmaceutically acceptable buffer solution may have a basic pH, e.g., a pH ranging from about 9 to about 11, such as a pH of about 10. The second pharmaceutically acceptable buffer solution may be pH adjusted with a suitable base (e.g., sodium hydroxide).

The pharmaceutically acceptable buffer solution(s) may further comprise one or more materials and/or agents. As mentioned above, the plurality of PEG particles and plurality of collagen particles may be combined with a buffer solution to form a slurry. The buffer solution of the slurry may have an acidic pH. Thus, for example, when the composition or kit comprises two pharmaceutically acceptable buffer solutions, the first pharmaceutically acceptable buffer solution having an acidic pH may comprise the PEG particles and collagen particles. The resulting slurry may have a pH ranging from about 3 to about 5, such as a pH of about 4.

Exemplary agents that may be included in the pharmaceutically acceptable buffer solution(s) and/or dry particulate mixture (and gel compositions prepared from such buffer solution(s)) and particulate mixture include, but are not limited to, crosslinking agents, antimicrobial agents, anti-inflammatory agents, and radiopaque materials.

Exemplary crosslinking agents useful for the present disclosure include, but are not limited to, trilysine acetate, N-(3-dimethylaminopropyl)-N′-ethylcarbodiimide hydrochloride, N-hydroxysuccinimide, and combinations thereof. In some examples, the first pharmaceutically acceptable buffer solution may comprise at least one crosslinking agent. In some examples, the first pharmaceutically acceptable buffer solution may comprise at least one crosslinking agent in combination with the plurality of PEG particles and the plurality of collagen particles, e.g., forming a slurry that further comprises at least one crosslinking agent.

Antimicrobial agents may be useful to impart antimicrobial properties (e.g., antibiotic properties) and/or mechanical properties, among other properties. In some cases, antimicrobial agents may be released slowly from the composition into surrounding tissue and other anatomy during the healing process to minimize risk of infection reoccurring and/or to combat residual infection. Exemplary antimicrobial agents useful for the present disclosure include, but are not limited to, ciprofloxacin, augmentin, metal ions, metal oxides, and combinations thereof. Metal ions and metal oxides may impart the gel compositions with other properties that could be useful for tissue repair, such as magnetism, wound healing, and/or electrical conductivity. The metal ion(s) or metal oxide(s) may comprise, for example, silver, gold, zinc, titanium, magnesium, or copper. In some examples, the dry particulate mixture comprises at least one antimicrobial agent. In some examples, a slurry comprising the particulate mixture and a buffer solution (e.g., the first pharmaceutically acceptable buffer solution) comprises at least one antimicrobial agent.

As mentioned above, the PEG particles may comprise one or more functional groups that are radiopaque, e.g., compounds comprising iodine such as 3,4,5-triiodobenzoic acid. Additionally or alternatively, the first pharmaceutically acceptable buffer solution or second pharmaceutically acceptable buffer solution may comprise a radiopaque material, e.g., a compound with a radiopaque moiety. Radiopacity of the gel may assist medical professionals with viewing and/or confirming the location of the gel at the target site, such as a fistula, during imaging of a subject.

The compositions herein, e.g., gels and hydrogels, may be formed by combining the plurality of PEG particles, the plurality of collagen particles, and the pharmaceutically acceptable buffer solution(s). As mentioned above, the PEG polymer of the PEG particles may include one or more functional groups available for crosslinking. Without being bound to any particular theory, while in an acidic buffer solution, the PEG polymer may initially remain in the form of a slurry, e.g., together with collagen particles, due at least in part to the pH of the buffer solution. For example, the relatively low pH may inhibit or slow crosslinking of the PEG polymer. Once combined with a basic buffer solution, the change in pH may initiate and/or accelerate crosslinking of the PEG polymer to form a gel (e.g., with collagen particles embedded therein). The gel may form relatively quickly, such as within 1 minute, within 45 seconds, within 30 seconds, within 20 seconds, or within 15 seconds of combining a first pharmaceutically acceptable buffer solution (comprising PEG particles and collagen particles) at an acidic pH with a second pharmaceutically acceptable buffer solution at a basic pH.

The first pharmaceutically acceptable buffer solution and second pharmaceutically acceptable buffer solution may be formulated for injection and mixing at a target site, such as a fistula. Thus, for example, a slurry comprising the first pharmaceutically acceptable buffer solution, PEG particles, and collagen particles may be combined with the second pharmaceutically acceptable buffer solution at a target site of the subject to form a gel at the target site, wherein the gel forms within 20 to 30 seconds after combination and/or administration and may fill the target site. In some examples, the target site may be a fistula of a gastrointestinal tract of the subject. In some examples, the target site may be a legion or polyp of a gastrointestinal tract of the subject. For example, the fistula, legion, or polyp may be within the esophagus, stomach, small intestine, large intestine, or colon (including, e.g., the anus). Once formed, the gel may have a viscosity that allows the gel to set and adhere to tissue (e.g., mucosa or submucosa) of the target site. It will be appreciated that the gel may be biocompatible, biodegradable, and/or bioresorbable, e.g., to prevent triggering an immune response from the subject.

The gel may be formulated to support tissue repair while degrading at a rate such that removal of the gel following formation of new tissue and/or vascularization of tissue is not necessary. For example, the gel may be formulated to degrade concurrently with tissue repair at the same or similar rate as the body of the subject's healing timescale (e.g., tissue repair duration). In these cases, the gel may facilitate repair of damaged tissue with reduced fibrotic scarring and/or promote accelerated tissue regeneration and vascularization of the regenerated tissue. Further, for example, the gel may help to draining infection from the target site, e.g., treating and/or eradicating a fistula, to avoid persistent or recurrent disease while preserving functionality of surrounding anatomy (e.g., preserving the anal sphincter function in the case of a fistula of the colon). When the gel comprises an antimicrobial agent, the antimicrobial agent may be released to tissue surrounding the target site during degradation of the gel to further inhibit or prevent infection.

Additionally, in some examples, by forming a non-permeable barrier, the gel may at least partially inhibit or completely prevent ingress of undesirable materials to the target site such as a fistula. This barrier may facilitate closure of the fistula. At the same time, the gel may allow migration of biological components involved in wound healing and/or tissue repair, such as fibroblasts and endothelial cells. Thus, for example, collagen present in the gel may further promote healing and tissue repair assisted by such biological components. The gel may provide a regenerative scaffold for host fibroblasts to migrate to the target site to promote tissue healing and/or repair damaged tissue with reduced fibrotic scarring.

### Kits

In some aspects of the present disclosure, the compositions herein may be preparing from components of a kit. For example, a medical professional may use the kit to prepare a gel at a target site of a subject, in situ.

The kit may comprise, for example, a particulate mixture (e.g., a dry particulate mixture) comprising, consisting essentially of, or consisting of the plurality of PEG particles and the plurality of collagen particles, a first pharmaceutically acceptable buffer solution, and a second pharmaceutically acceptable buffer solution. Any of the dry particulate mixture, first pharmaceutically acceptable buffer solution, and/or second pharmaceutically acceptable buffer solution may further comprise at least one of a crosslinking agent, an antimicrobial agents, or radiopaque material. Optionally, the kit may further comprise instructions and/or a delivery device, such as a double-barreled syringe. Each of the dry particulate mixture, the first pharmaceutically acceptable buffer solution, and the second pharmaceutically acceptable buffer solution may be provided in separate containers. In some examples, the kit may comprise a container that includes the particulate mixture of PEG particles and collagen particles and the first pharmaceutically acceptable buffer solution, e.g., as a slurry. In such cases, the slurry and the second pharmaceutically acceptable buffer solution may be provided in separate containers.

As mentioned above, the weight ratio of the plurality of PEG particles to the plurality of collagen particles in the dry particulate mixture or slurry may range from about 10:1 to about 4:1, e.g., about 8:1 to about 6:1. Thus, for example, the dry particulate mixture or slurry may comprise from about 1.75 g to about 3.25 g PEG particles (e.g., about 2 g to about 3 g, or about 2.25 g to about 2.75 g) and from about 0.05 g to about 0.9 g collagen particles (e.g., about 0.08 g to about 0.7 g, or about 0.2 g to about 0.5 g).

In some aspects, the kits herein may further comprise instructions for preparing the composition and/or administering the composition to a target site of a subject. In some examples, the instructions may include combining a dry particulate mixture with a first pharmaceutically acceptable buffer solution and a second pharmaceutically acceptable buffer solution to form a gel at a target site, e.g., for application to a fistula. In examples where the kit includes a delivery device such as a double-barrel syringe, instructions may include instructions for combining the dry particulate mixture with the first pharmaceutically acceptable buffer solution to form a slurry, introducing the slurry into a first barrel of the syringe, and introducing the second pharmaceutically acceptable buffer solution into a second barrel of the syringe.

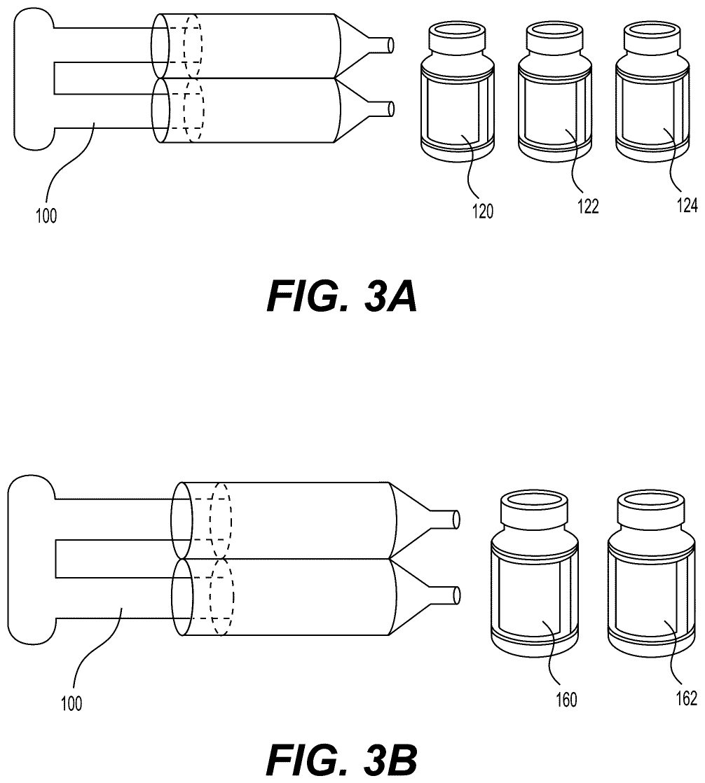

Exemplary components of kits are illustrated in FIGS. 3A and 3B, including delivery device 100 such as a double-barreled syringe. The delivery device 100 may be configured for use with an endoscope. For example, the delivery device may include a mixing head capable of administering the contents of each barrel at the same flow rate or substantially the same flow rate, e.g., via respective tubing, through the working channel of an endoscope for delivery to the target site.

FIG. 3A shows an example comprising the delivery device 100 with three containers 120, 122, 124 (e.g., three vials), wherein container 120 includes a dry particulate mixture of PEG particles and collagen particles, container 122 includes a first pharmaceutically acceptable buffer solution at acidic pH, and container 124 includes a second pharmaceutically acceptable buffer solution at basic pH. FIG. 3B shows an example comprising the delivery device 100 with two containers 160, 162 (e.g., two vials), wherein container 160 includes a slurry comprising PEG particles, collagen particles, and a first pharmaceutically acceptable buffer solution at acidic pH, and container 162 includes a second pharmaceutically acceptable buffer solution at basic pH. Other examples of kits according to the present disclosure do not include a delivery device. Any of the kits herein may include instructions for preparing and/or administering the composition to a target site of a subject.

The kits herein may be prepared combining the plurality of collagen particles with the plurality of PEG particles to obtain a dry particulate mixture, packaging the dry particulate mixture with the first pharmaceutically acceptable buffer solution and the second pharmaceutically acceptable buffer solution. In some examples, the plurality of collagen particles may be prepared from a collagen scaffold, e.g., by grinding the collagen scaffold into particles of the desired size, as discussed below.

### Methods

According to some aspects of the present disclosure, the plurality of collagen particles may be prepared from a collagen scaffold, e.g., to provide the collagen particles with porosity to facilitate tissue repair. The collagen scaffold may be ground into particles that retain porosity. For example, collagen may be hydrated in a weakly acidic solution (e.g., acetic acid) to produce a slurry and subsequently dried (e.g., freeze-dried) to form a porous scaffold. Optionally, the collagen slurry may include a crosslinking agent, which may ultimately provide for a sturdier gel. Conditions, such as freeze-drying conditions, during manufacture and processing of the collagen scaffold may be controlled to provide for the desired porosity. The collagen scaffold may have a sponge-like structure and/or sponge body due to the plurality of pores formed therein. In some examples, the collagen scaffold may have an average pore size ranging from about 50 μm to about 250 μm, such as from about 100 μm to about 200 μm. Average pore size may be measured by a suitable technique, such as by measure pore size optically by scanning electron microscopy (SEM) and/or using analytical software in combination with SEM. The collagen scaffold may be ground into a plurality of collagen particles, e.g., having an average particle size ranging from about 200 μm to about 600 μm, such as about 250 μm to about 550 μm, or about 300 μm to about 500 μm.

In some aspects of the present disclosure, the compositions herein may be formed by combining a plurality of collagen particles with a plurality of PEG particles to form a dry particulate mixture (which may be packaged in a container such as a vial). A first pharmaceutically acceptable buffer solution having a pH of 4 and second pharmaceutically acceptable buffer solution having a pH of 10 may be stored separately in respective container. Any of the dry particulate mixture, first pharmaceutically acceptable buffer solution, and/or second pharmaceutically acceptable buffer solution may further comprise at least one of a crosslinking agent, an antimicrobial agents, or radiopaque material. The composition may be formed at the target site, such as a fistula, by combining the dry particulate mixture with the buffer solutions at the target site as described above. The target site may be scoped and/or irrigated to prepare for the administration of the composition. The subject may be rotated to allow gravity to assist with placement of the composition within the area of the target site. In some cases, the dry particulate mixture may be combined with the first pharmaceutically acceptable buffer solution, e.g., to form a precursor slurry. The precursor slurry and second pharmaceutically acceptable buffer solution (e.g., acting as an accelerator buffer solution) at the same or substantially the same flow rate to the target site. The precursor slurry and accelerator buffer solution may be administered until the target site is substantially or completely filled with the composition. Once the precursor solution and accelerator buffer solution are combined at the target site, the composition may form into a gel, such as a hydrogel, within about 30 seconds, within about 20 seconds, or within about 15 seconds, setting and adhering to tissue (e.g., mucosa or submucosa) of the target site. Once the gel sets and adheres, the gel may inhibit or prevent ingress of undesired materials while allowing passage of biological components to assist with tissue repair, as discussed above. If the target site is an anal fistula, for example, the gel may inhibit or prevent ingress of fecal material to the anal fistula.

The compositions herein may help to limit recovery time. For example, in some cases wherein the target site is a fistula, closing of the fistula may occur within about 2 months to about 6 months, e.g., about 2 months to about 4 months, about 2.5 months to about 3.5 months, or as about 3 months. The subject's body may absorb the gel over time, e.g., within about 5 months to about 7 months, such as about 6 months. In some cases (e.g., in examples in which the composition does not include an antimicrobial agent), an antibiotic or other antimicrobial agent may be administered to inhibit or prevent infection at the target site.

Other aspects of the present disclosure will be apparent to those skilled in the art from consideration of the specification and practice of the examples disclosed herein. It is intended that the specification and examples be considered as exemplary only, with a true scope and spirit of the present disclosure being indicated by the following claims.

What is claimed is:

1. A kit for treating a fistula, the kit comprising:
a dry particulate mixture comprising:
a plurality of polyethylene glycol particles; and
a plurality of collagen particles;
a first pharmaceutically acceptable buffer solution having a pH ranging from about 3 to about 5; and
a second pharmaceutically acceptable buffer solution having a pH ranging from about 9 to about 11.

2. The kit of claim 1, wherein the first pharmaceutically acceptable buffer solution comprises at least one crosslinking agent.

3. The kit of claim 2, wherein the at least one crosslinking agent comprises from trilysine acetate, N-(3-dimethylaminopropyl)-N′-ethylcarbodiimide hydrochloride, or N-hydroxysuccinimide.

4. The kit of claim 1, further comprising instructions for combining the dry particulate mixture with the first pharmaceutically acceptable buffer solution and the second pharmaceutically acceptable buffer solution to form a gel for application to a fistula.

5. The kit of claim 4, further comprising a double-barreled syringe, wherein the instructions combining the dry particulate mixture with the first pharmaceutically acceptable buffer solution to form a slurry, introducing the slurry into a first barrel of the syringe, and introducing the second pharmaceutically acceptable buffer solution into a second barrel of the syringe.

6. The kit of claim 1, wherein a weight ratio of the plurality of polyethylene glycol particles to the plurality of collagen particles in the dry particulate mixture ranges from about 8:1 to about 6:1.

7. The kit of claim 1, wherein the plurality of collagen particles has an average particle size ranging from about 300 μm to about 500 μm.

8. The kit of claim 1, wherein the plurality of polyethylene glycol particles comprises a polyethylene glycol polymer comprising one or more functional groups chosen from carboxylic acid groups, ester groups, amine groups, or a combination thereof.

9. The kit of claim 8, wherein the one or more functional groups comprise succinimide groups.

10. The kit of claim 1, wherein the first pharmaceutically acceptable buffer solution or the second pharmaceutically acceptable buffer solution comprises a radiopaque material, an antimicrobial agent, or both a radiopaque material and an antimicrobial agent.

11. A method of treating a subject, the method comprising:
combining a dry particulate mixture with a first pharmaceutically acceptable buffer solution having a pH ranging from about 3 to about 5 to form a slurry, wherein the dry particulate mixture comprises a plurality of polyethylene glycol particles and a plurality of collagen particles; and
combining the slurry with a second pharmaceutically acceptable buffer solution having a having a pH ranging from about 9 to about 11 at a target site of the subject to form a gel at the target site.

12. The method of claim 11, wherein the plurality of collagen particles has an average particle size ranging from about 300 μm to about 500 μm.

13. The method of claim 11, wherein the first pharmaceutically acceptable buffer solution comprises a crosslinking agent.

14. The method of claim 11, wherein the target site is a fistula of a gastrointestinal tract of the subject.

15. The method of claim 11, wherein the gel forms within 30 seconds of combining the slurry with the second pharmaceutically acceptable buffer solution at the target site.

16. The method of claim 11, wherein the plurality of polyethylene glycol particles comprises a polyethylene glycol polymer comprising one or more functional groups chosen from carboxylic acid groups, ester groups, amine groups, or a combination thereof.

17. A method of treating a subject, the method comprising:
administering, to a target site of the subject, a slurry comprising:
a plurality of polyethylene glycol particles;
a plurality of collagen particles; and
a first pharmaceutically acceptable buffer solution having a pH ranging from about 3 to about 5; and
administering, to the target site, a second pharmaceutically acceptable buffer solution having a pH raging from about 9 to about 11 to combine with the slurry;
wherein the slurry and the second pharmaceutically acceptable buffer solution form a gel at the target site within 30 seconds of being combined at the target site; and
wherein the target site is a fistula of a gastrointestinal tract of the subject.

18. The method of claim 17, wherein the slurry and the second pharmaceutically acceptable buffer solution are administered to the target site at the same time.

19. The method of claim 17, wherein the plurality of polyethylene glycol particles comprises a polyethylene glycol polymer comprising one or more functional groups chosen from carboxylic acid groups, ester groups, amine groups, or a combination thereof.

20. The method of claim 17, wherein the slurry and the second pharmaceutically acceptable buffer solution are administered using a double-barreled syringe, wherein a first barrel of the syringe comprises the slurry and a second barrel of the syringe comprises the second pharmaceutically acceptable buffer solution.
